(19) United States
(12) Patent Application Publication
(10) Pub. No.: US 2012/0003669 A1
(43) Pub. Date: Jan. 5, 2012

(54) Immunohistochemical staining method and immunohistochemical staining apparatus

(76) Inventors: Yoshihiro MINAMIYA, Akita-shi (JP); Hiroshi Toda, Akita-shi (JP); Jun-ichi Ogawa, Akita-shi (JP); Yoichi Akagami, Akita-shi (JP); Masami Kagaya, Akita-shi (JP)

(21) Appl. No.: 13/151,730

(22) Filed: Jun. 2, 2011

(30) Foreign Application Priority Data
Jul. 2, 2010 (JP) 2010-151695

(51) Int. Cl. G01N 33/53; C12M 1/34; G01N 21/64

(52) U.S. Cl. 435/7.9; 435/7.1; 435/288.7

(57) ABSTRACT

An immunohistochemical staining apparatus for carrying out immunohistochemical staining includes a sample chamber (1) in which a primary sample (s1) comprised of a tissue specimen (t) to which a primary antibody (Ig1) is applied is disposed between a sample-side electrode (11) and an opposed electrode (12). The sample chamber (1) has first stirring means for noncontactlessly stirring the primary sample (s1) by applying for a first prescribed time period an alternating electric field adjusted so that the opposed electrode (12) is minus with respect to the sample-side electrode (11), and second stirring means for noncontactlessly stirring a secondary sample (s2) comprised of the noncontactlessly-stirred primary sample (s1) to which a labeled secondary antibody (Ig2) is applied by applying for a second prescribed time period an alternating electric field adjusted so that the opposed electrode (12) is minus with respect to the sample-side electrode (11).

### BACKGROUND OF THE INVENTION

1. Field of the Invention

The present invention relates to an immunohistochemical staining method and apparatus for revealing the expression and localization of specific proteins in biological tissue. In particular, it relates to an immunohistochemical staining method and apparatus that can speed up a series of operations, for example, it can be utilized in rapid intraoperative pathological diagnoses based on the detection of cancer-specific proteins during operations, and can also reduce costs by making it possible to economize on the use of antibodies.

2. Background Art

As a method used during a surgical operation for deciding how to proceed, there is known rapid intraoperative pathological diagnosis. Since rapid intraoperative pathological diagnosis is conducted by temporarily stopping the operation, which imposes strict time constraints, conventionally the pathology has been decided based on the morphological features of the biological tissue, which can often cause misdiagnoses. There is therefore a need for improved diagnostic precision.

In the case of rapid intraoperative pathological diagnosis of breast cancer or malignant melanoma, for example, the sentinel lymph node, which is the closest lymph node to which lymph fluid from the cancerous lesion flows, is used to confirm morphologically the presence or absence of metastasis, and to decide whether or not to excise lymph nodes that are further from the lesion than the sentinel lymph node. Although in this rapid intraoperative pathological diagnosis using the sentinel lymph node the pathological diagnosis is based on the morphology of the tissue stained with HE (hematoxylin and eosin), there are cases in which micrometastasis to the lymph node has been overlooked.

As a means for revealing the expression and localization of specific proteins in biological tissue, there is known the immunohistochemical staining method. Immunohistochemical staining makes it possible to make a diagnosis with very high precision by using a highly-specific antibody to detect a specific protein. However, following the sampling of the target tissue, the conventional immunohistochemical staining method involves a sequence of steps including fixing, washing and blocking the specimen, an antigen-antibody reaction using a primary antibody, an antigen-antibody reaction using a secondary antibody, color development using a chromogenic solution, and so forth, as shown in FIG. 6, for example, that requires at least two hours, making it unsuitable for rapid intraoperative pathological diagnosis in which there are strict time constraints.

However, the strong need to make use of immunohistochemical staining in rapid intraoperative pathological diagnosis has led to proposals, such as that of the invention disclosed by JP HEI 8-304388 (A) (Patent Literature 1), which endeavors to shorten the time required for the sequence of immunohistochemical staining steps by utilizing ultrasonic stirring technology.

In JP 2010-119388 (A) (Patent Literature 2), the present inventors disclosed a method they developed to shorten the time required for reactions, such as the nucleic acid hybridization reaction and the ELISA reaction taking place in minute drops of solution in the order of microliters, by applying a high-voltage alternating electric current.

However, in the invention disclosed by the above Patent Literature 1, cavitation caused by the use of ultrasonic waves of a frequency of 20 kHz to 40 kHz to stir tissue specimens causes a rise in temperature, making it unsuitable for the detection of proteins which are sensitive to changes of temperature and the like. Moreover, there was a concern that the dispersion of tissue specimens caused by the cavitation could make it impossible to implement the immunohistochemical staining. There was also a concern that the rapid stirring by the ultrasonic waves could cause a sudden rise in molecular collisions that would degrade and damage the protein, reducing the accuracy of the immunohistochemical staining. Another problem was the noise caused by the use of ultrasonic waves. Therefore, the concern was that problems in the implementation and the ultrasonic noise made it difficult to utilize in rapid intraoperative pathological diagnosis.

An object of the present invention is therefore to provide an immunohistochemical staining method and immunohistochemical staining apparatus that can be applied to rapid intraoperative pathological diagnosis, which has strict time constraints, can greatly improve diagnostic precision and, by markedly reducing the amount of antibodies used in the immunohistochemical staining, can also reduce the cost.

### DISCLOSURE OF THE INVENTION

As a result of assiduous studies conducted to attain the above object, it was discovered that by using an immunohistochemical staining method in which a high-voltage fluctuating electric field was applied in a prescribed orientation, and in which a secondary antibody was used in addition to a primary antibody, it was possible to rapidly detect an antigen and, moreover, to markedly reduce the amount of antibody used, thereby leading to this invention.

That is, in accordance with the immunohistochemical staining method of this invention, a primary sample is formed by applying a primary antibody to a tissue specimen disposed between a sample-side electrode and an opposed electrode, and an alternating electric field adjusted so that the opposed electrode is minus with respect to the sample-side electrode is applied for a first prescribed time period to noncontactlessly stir the primary sample, after which a secondary sample is formed by applying a labeled secondary antibody to the primary sample, an alternating electric field adjusted so that the opposed electrode is minus with respect to the sample-side electrode is applied for a second prescribed time period to noncontactlessly stir the secondary sample, and a chromogenic solution is applied to stain the tissue specimen.

Preferably the alternating electric field is a square wave superposed with a signal of a frequency of from 10 Hz to 300 Hz, and the strength of the applied alternating electric field is preferably 0.35 to 2.50 kV/mm. Preferably the first prescribed time period for which the alternating electric field is applied is 60 minutes to 180 minutes, and/or the second prescribed time period for which the alternating electric field is applied is 30 seconds to 5 minutes. The first prescribed time period for which the alternating electric field is applied may also be 1 minute to 5 minutes, and/or the second prescribed time period for which the alternating electric field is applied 30 seconds to 5 minutes. The concentration of the applied primary antibody is 0.25 to 1.0 μg/mL when it is desired to use the antibody economically, and 4.0 to 6.0 μg/mL when it is desired to promote a rapid reaction. The labeling is preferably done using any selected from an enzyme label, a fluorescence label, a radioisotope label, and a gold colloid particle label.

The immunohistochemical staining apparatus of the invention has a sample chamber in which a primary sample comprised of a tissue specimen to which a primary antibody is applied is disposed between a sample-side electrode and an opposed electrode. The sample chamber includes first stirring means for noncontactlessly stirring the primary sample by applying for a first prescribed time period an alternating electric field adjusted so that the opposed electrode is minus with respect to the sample-side electrode, and second stirring means for noncontactlessly stirring a secondary sample comprised of the noncontactlessly-stirred primary sample to which a labeled secondary antibody is applied by applying for a second prescribed time period an alternating electric field adjusted so that the opposed electrode is minus with respect to the sample-side electrode.

Preferably the alternating electric field is a square wave superposed with a signal of a frequency of from 10 Hz to 300 Hz, and the strength of the applied alternating electric field is preferably 0.35 to 2.50 kV/mm. Preferably the first prescribed time period for which the alternating electric field is applied is 60 minutes to 180 minutes, and/or the second prescribed time period for which the alternating electric field is applied is 30 seconds to 5 minutes. The first prescribed time period for which the alternating electric field is applied is also 1 minute to 5 minutes, and/or the second prescribed time period for which the alternating electric field may be applied 30 seconds to 5 minutes. The concentration of the applied primary antibody is 0.25 to 1.0 μg/mL when it is desired to use the antibody economically, and 4.0 to 6.0 μg/mL when it is desired to promote a rapid reaction.

The same means may be used for the first stirring means and the second stirring means in the immunohistochemical staining apparatus of the present invention. Also, it is desirable to use a configuration in which a part of the opposed electrode that is directly above the tissue specimen mounted in the sample chamber is provided with a projecting portion.

In accordance with the immunohistochemical staining method of the invention, a primary sample is formed by applying a primary antibody to a tissue specimen disposed between a sample-side electrode and an opposed electrode, and noncontactlessly stirring the primary sample by applying for a first prescribed time period an alternating electric field adjusted so that the opposed electrode is minus with respect to the sample-side electrode; then forming a secondary sample is formed by applying a labeled secondary antibody to the primary sample, and contactlessly stirring the secondary sample by applying for a second prescribed time period an alternating electric field adjusted so that the opposed electrode is minus with respect to the sample-side electrode.

Thus, the noncontact stirring energizes the antigen-antibody reaction, thereby making the method applicable to rapid intraoperative pathological diagnoses, which has strict time constraints. Also, since the antigen-antibody reaction can be energized by the contact stirring, the amount of antibody used in the immunohistochemical staining can be markedly reduced, making it possible to reduce the cost of the immunohistochemical staining method which is said to depend on the price of the primary antibody.

Generating the alternating electric field as a square wave superposed with a signal having a frequency below the audible range provides stirring that is gentle and efficient and does not cause cavitation problems. The strength of the applied alternating electric field is 0.35 to 2.50 kV/mm, so there are no electrical discharges, again providing gentle stirring for efficient immunohistochemical staining. Making the first prescribed time period for which the alternating electric field is applied 60 to 180 minutes and/or the second prescribed time period for which the alternating electric field is applied 30 seconds to 5 minutes makes it possible to carry out the immunohistochemical staining under conditions that markedly reduce the amount of the primary antibody used. Also, making the first prescribed time period for which the alternating electric field is applied 1 to 5 minutes and/or the second prescribed time period for which the alternating electric field is applied 30 seconds to 5 minutes makes it possible to apply the invention to rapid intraoperative pathological diagnosis, which has strict time constraints. Using the applied primary antibody at a concentration of 0.25 to 1.0 μg/mL markedly reduces the amount of primary antibody used, helping to reduce the cost. Using a concentration of 4.0 to 6.0 μg/mL makes the invention applicable to the strict time constraints of rapid intraoperative pathological diagnoses.

The immunohistochemical staining apparatus of the invention has a sample chamber in which a primary sample comprised of a tissue specimen to which a primary antibody is applied is disposed between a sample-side electrode and an opposed electrode. The sample chamber includes first stirring means for noncontactlessly stirring the primary sample by applying for a first prescribed time period an alternating electric field adjusted so that the opposed electrode is minus with respect to the sample-side electrode, and second stirring means for noncontactlessly stirring a secondary sample comprised of the noncontactlessly-stirred primary sample to which a labeled secondary antibody is applied by applying for a second prescribed time period an alternating electric field adjusted so that the opposed electrode is minus with respect to the sample-side electrode.

The sample chamber is also provided with dropper means for applying chromogenic solution to the noncontactlessly-stirred secondary sample. Thus the effect of the immunohistochemical staining method can be provided by an immunohistochemical staining apparatus that is fully-equipped for the purpose.

Generating the alternating electric field as a square wave superposed with a signal having a frequency below the audible range provides stirring that is gentle and efficient and does not cause cavitation problems. The strength of the applied alternating electric field is 0.35 to 2.50 kV/mm, so there are no electrical discharges, again providing gentle stirring for efficient immunohistochemical staining. Making the first prescribed time period for which the alternating electric field is applied 60 to 180 minutes and/or the second prescribed time period for which the alternating electric field is applied 30 seconds to 5 minutes makes it possible to carry out the immunohistochemical staining under conditions that markedly reduces the amount of the primary antibody used. Also, making the first prescribed time period for which the alternating electric field is applied 1 to 5 minutes and/or the second prescribed time period for which the alternating electric field is applied 30 seconds to 5 minutes makes it possible to apply the invention to rapid intraoperative pathological diagnosis, which has strict time constraints. Also, using the applied primary antibody at a concentration of 0.25 to 1.0 μg/mL markedly reduces the amount of primary antibody used, helping to reduce the cost, while using a concentration of 4.0 to 6.0 μg/mL makes the invention applicable to the strict time constraints of rapid intraoperative pathological diagnoses. Also, an antibody concentration of 1.0 to 4.0 μg/mL can be used when rapid detection is required while also economizing on the amount of antibody used.

The immunohistochemical staining apparatus of the invention can be made smaller and simpler by using the same means for the first and second stirring means. Providing the part of the opposed electrode that is directly above the tissue specimen with a projecting portion enables the strength of the alternating electric field to be constrained, ensuring that the generation of electrical discharges is prevented.

With this invention, an alternating electric field is applied to a tissue specimen mounted between a sample-side electrode and an opposed electrode, with the alternating electric field being adjusted so that the opposed electrode is minus with respect to the sample-side electrode, enabling the antigen-antibody reaction between the tissue specimen and each of the antibodies to proceed under noncontact stirring generated by the Coulomb force of the alternating electric field. This gives rise to molecular collisions which, by producing an environment that facilitates the action of the van der Waals force, speeds up the antigen-antibody reactions. Also, since the invention does not use rotors or stirring elements it is difficult for contamination to occur, process time is reduced and variation is suppressed, providing clear results. Another advantage is that it does not generate the noise comparing with ultrasonic stirring. The apparatus has a simple configuration that makes it easy to reduce the size, and if transparent electrodes are used, the progress of the chromogenic reaction of a solution can be observed as it takes place. Also, the stirring is not subject to limitations under various environment conditions such as temperature, humidity, vacuum and gaseous atmospheres.

### BRIEF DESCRIPTION OF THE DRAWINGS

FIG. 1 is a schematic diagram for explaining the immunohistochemical staining apparatus of the invention.

FIG. 2 is an explanatory diagram relating to the overlapping frequencies of the alternating electric field in the immunohistochemical staining apparatus of the invention.

FIG. 3 is a block diagram for explaining the protocol of the immunohistochemical staining method of the invention, with a) being the antibody economy method and b) the rapid method.

FIG. 4 is an explanatory diagram for explaining cancer tissue stained using the immunohistochemical staining method of the invention.

FIG. 5 is an explanatory diagram of another immunohistochemical staining apparatus according to the invention.

FIG. 6 is a block diagram for explaining the protocol of a conventional immunohistochemical staining method.

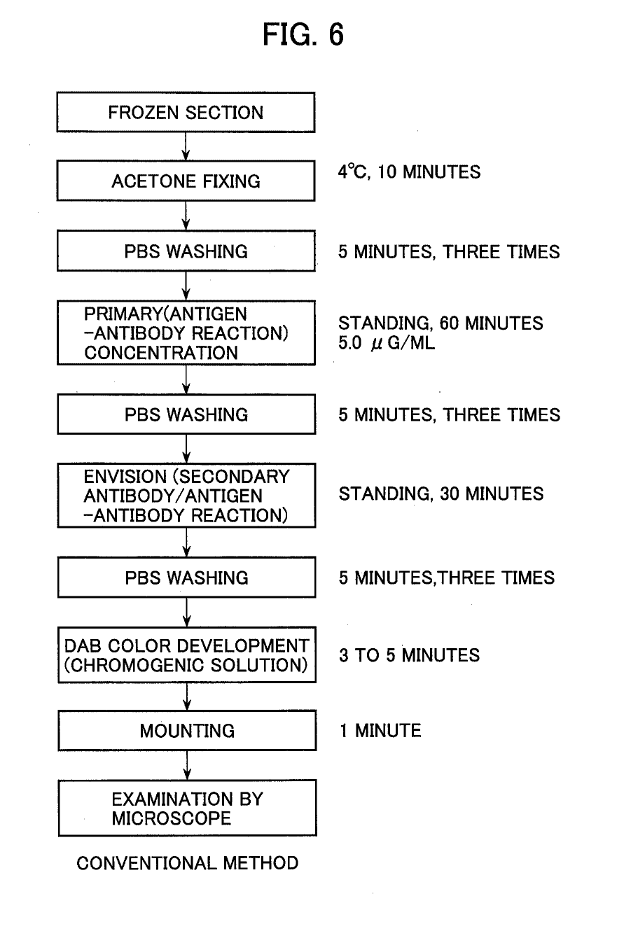

### DESCRIPTION OF THE PREFERRED EMBODIMENTS

Embodiments of the present invention will now be described in the following with reference to the drawings.

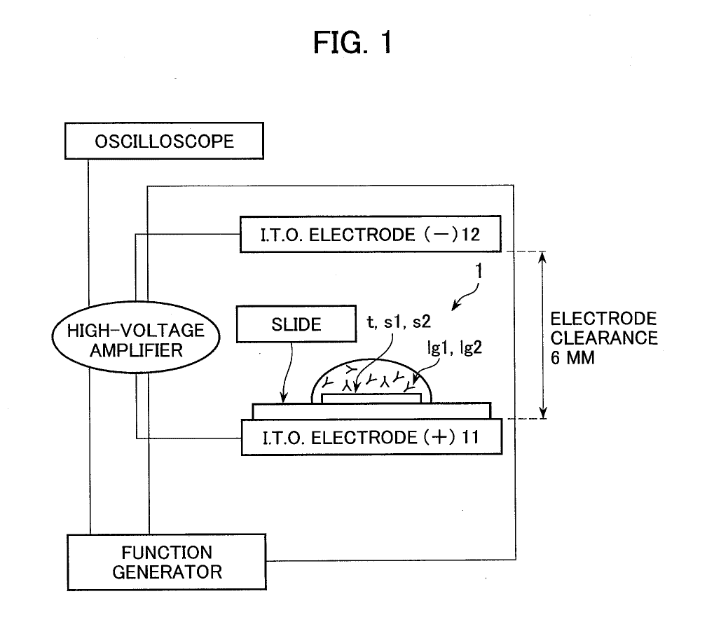

FIG. 1 shows an example of an immunohistochemical staining apparatus according to the present invention. As shown in the drawing, the apparatus includes a sample chamber 1 in which a tissue specimen t fixed to a microscope slide is disposed between a sample-side electrode 11 formed of indium tin oxide (ITO), for example, and an opposed electrode 12. In particular, an alternating electric field is generated in the sample chamber 1 in which the opposed electrode 12 is minus with respect to the sample-side electrode 11. The alternating electric field functions as first and second stirring means for noncontact stirring of primary antibody Ig1 and secondary antibody Ig2 dripped onto the tissue specimen t. In this example, the clearance between the electrodes 11 and 12 is 6 mm. In addition, the part of the opposed electrode 12 that is directly above the tissue specimen t in the sample chamber 1 has a projecting portion that projects towards the tissue specimen t, forming what is a preferred configuration from the standpoint of constraining the field strength of the alternating electric field and minimizing the possibility of electrical discharges occurring. During operation, the interior of the sample chamber 1 forms a thermally sealed space. Also, while not shown, the sample chamber 1 is preferably provided with drip means for applying a chromogenic solution to the tissue specimen t mounted therein.

In the immunohistochemical staining apparatus according to this embodiment of the invention, the alternating electric field applied to the tissue specimen t is generated in a mode in which the electric waves are varied (having a burst waveform configuration). Specifically, under a humidity environment of 60±10%, the main applied voltage is generated by adding an offset voltage of 0.2 to 2.25 kV/mm to a square wave having an amplitude with a plus-side bias of 0.35 to 2.5 kV/mm onto which is externally superposed bundles of 2 to 40 frequencies of 0.1 to 800 Hz. There is a possibility that electrical discharges might occur if the applied electric field strength (kV/mm) has a large plus-side bias of more than 2.5 kV/mm, so it is adjusted so that the sum of the square-wave amplitude and added offset voltage does not result in a large plus-side bias of more than 2.5 kV/mm. Moreover, since there is a risk that stirring might not be generated if the applied electric field strength of the plus-side bias is smaller than 0.35 kV/mm, it is adjusted so that the sum of the square-wave amplitude and the added offset voltage does not result in a plus-side bias that is less than 0.35 kV/mm.

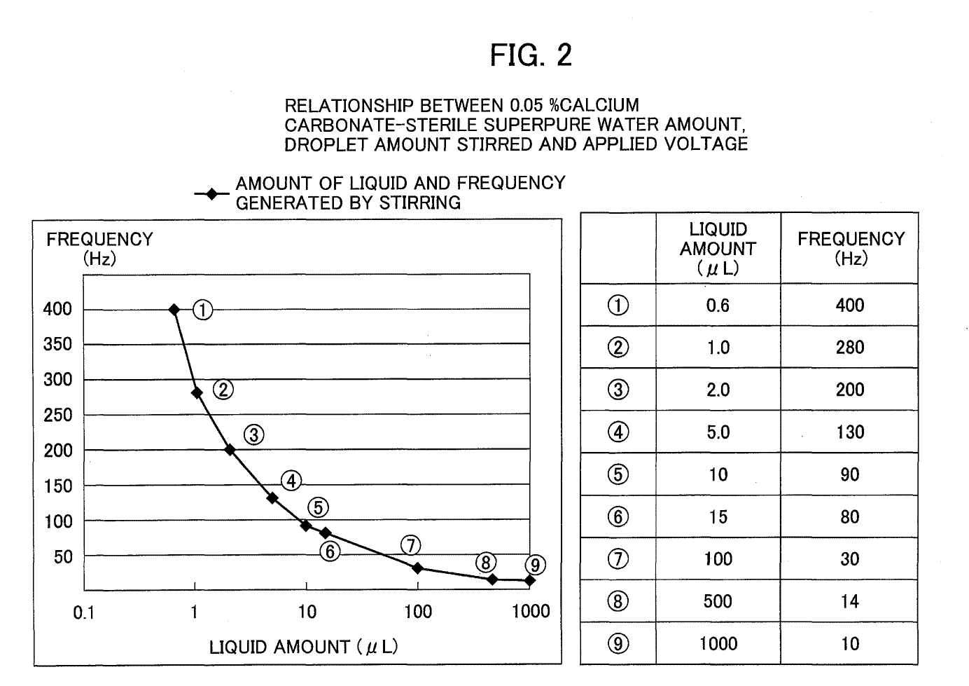

Noncontact stirring can thus be generated by externally superposing onto the alternating electric field bundles of 2 to 40 frequencies of from 0.1 to 800 Hz, and preferably from 10 to 400 Hz, as shown in FIG. 2. Taking into consideration the sample size, reagent amount and applied electric field used in normal immunohistochemical staining, frequencies of from 10 to 300 Hz are preferable. The number of superposed frequency oscillations may be adjusted within the above range in accordance with the amount of the liquid droplets (the amount of solution containing the primary antibody and the amount of solution containing the secondary antibody). The alternating electric field is biased to the plus side as that enables the electric field to draw in droplets, providing efficient stirring. A gentle oscillation can be delivered to the droplet wavefront by switching the alternating electric field on and off in bundles of about 2 to 20 cycles, a configuration that is preferred as it provides better stirring of the contents of the droplets.

Biological samples that can be used with this invention include biopsy specimens such as tumor tissues, cells and internal organs. These may be used as sections or, when necessary, fixed using paraformaldehyde or formalin or the like. Tissues can also be embedded in paraffin for sectioning. Frozen sections can be prepared by embedding specimens in a frozen mounting medium followed by rapid freezing using liquid nitrogen, for example, and a cryostat or the like used for the sectioning.

The secondary antibody used in the invention can be labeled using, for example, an enzyme label, a fluorescence label, a radioisotope label, or a gold colloid particle label. However, from the standpoint of detection sensitivity it is preferable to use an enzyme label.

Enzyme labeling substances that can be used include peroxidase, horseradish peroxidase, and alkaline phosphatase. When an enzyme label is used, the chromogenic solution that forms the substrate is dripped. Fluorescence labeling substances that can be used include fluorescein isothiocyanate and tetramethylrhodamine isothiocyanate, and radioisotope labeling substances that can be used include iodine radioisotopes such as ¹³¹I and ¹²⁵I.

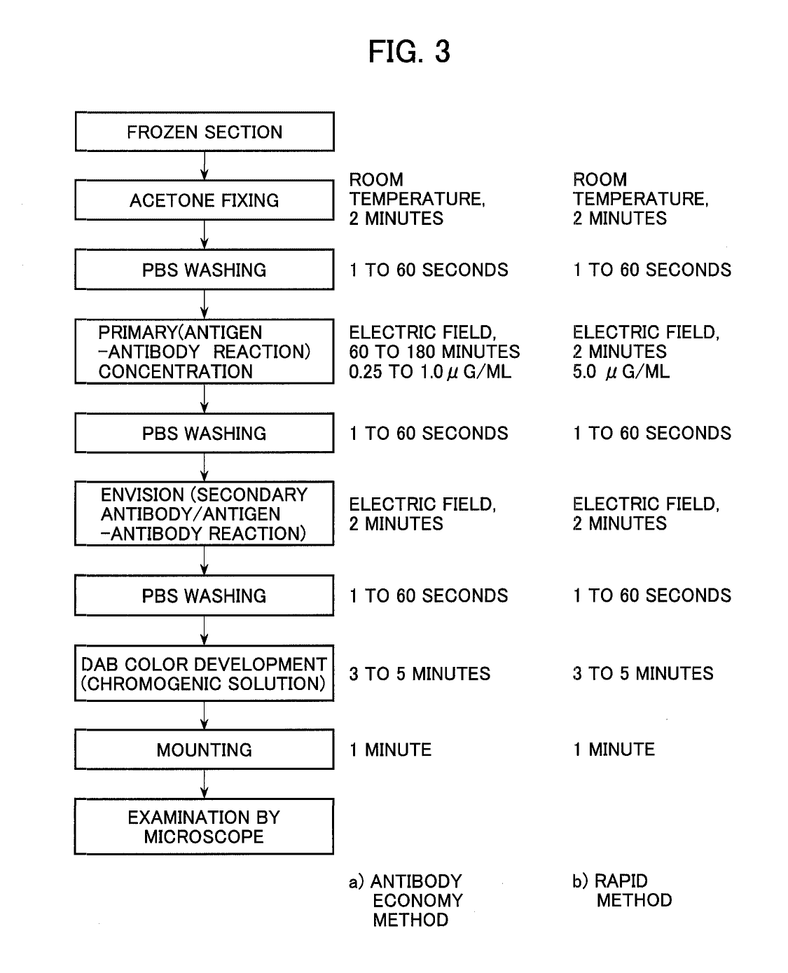

An example of the immunohistochemical staining method using the immunohistochemical staining apparatus according to the invention will now be described, with reference to FIG. 3. This example was carried out at an ambient temperature of 25±2° C. and a humidity of 60±10%.

A thin section of the tissue specimen t is placed on a microscope slide and fixed to the slide by immersion for 2 minutes at room temperature. The tissue specimen t fixed to the slide is then washed for about 30 seconds with phosphate buffered saline (PBS) having a suitable concentration.

Next, the tissue specimen t fixed to the slide is mounted in the sample chamber 1 of the immunohistochemical staining apparatus between the sample-side electrode 11 and opposed electrode 12, and a primary antibody Ig1 is dripped thereon to form a primary sample s1 fixed to the slide. Noncontact stirring of the primary antibody Ig1 is then carried out by application of an alternating electric field that is adjusted so the sample-side electrode 12 is minus with respect to the opposed electrode 11. The electric field is applied for 1 to 5 minutes when it is desired to carry out rapid immunohistochemical staining, and for 60 to 180 minutes when it is desired to carry out immunohistochemical staining in which the primary antibody Ig1 is used economically. The concentration of the applied primary antibody Ig1 is 4.0 to 6.0 μg/mL when it is desired to carry out rapid immunohistochemical staining, and 0.25 to 1.0 μg/mL when it is desired to carry out immunohistochemical staining in which the primary antibody Ig1 is used economically. The primary antibody Ig1 can be exemplified by, for example, an anti-cytokeratin antibody (AE1/AE3) that reacts with specific amino acid sequences of epithelial tissue cytokeratin.

In addition to anti-cytokeratin antibody (AE1/AE3), other primary antibodies that can be expected to be particularly effective as the primary antibody Ig1 used with the immunohistochemical staining apparatus of this invention include those that react with HER-2 specific amino acid sequences, CEA specific amino acid sequences, p53 specific amino acid sequences, and alpha-fetoprotein specific amino acid sequences.

Next, the primary sample s1 that has been noncontactlessly stirred is washed with PBS for about 1 to 60 seconds, the secondary antibody Ig2 is applied to the primary sample s1 to form a secondary sample s2 fixed to the slide, and noncontact stirring of the secondary sample s2 is then carried out by application for 2 minutes of an alternating electric field that is adjusted so the opposed electrode 11 is minus with respect to the sample-side electrode 12. A commercial antibody may be selected for the secondary antibody Ig2, while taking into consideration the combination formed with the primary antibody Ig1. In this example, an EnVision (Doko Company) product was used.

Next, the secondary sample s2 that has been noncontactlessly stirred is washed with PBS for about 1 to 60 seconds and is stained by applying drops of a chromogenic solution. A commercial chromogenic solution may be used, while taking into consideration the combination formed with the secondary antibody Ig2. In this example, an EnVision (Doko Company) peroxidase-conjugated product was used for the secondary antibody Ig2, so a diaminobenzedine (DAB) solution was used. Color development time was about 1 to 5 minutes.

Finally, a microscope is used to observe and check the state of color development of the secondary sample s2 in situ on the slide.

### EXAMPLES

Two examples of the immunohistochemical staining method using the immunohistochemical staining apparatus of the invention will now be described (see FIG. 3).

1) Antibody Economy Method

Animal or human epithelial cell tissue was frozen and embedded in OCT compound and a cryostat or other such cutting means used to cut a section of the tissue having a thickness of 5 to 10 μm. The section was placed on a microscope slide and fixed to the slide by immersion in acetone for 2 minutes at room temperature. The section on the slide was then washed with PBS for 30 seconds and the OCT compound was removed. A Doko Pen was used to draw a line around the periphery of the section to maintain the surface tension of the primary and secondary antibodies dripped thereon.

Anti-cytokeratin antibody (AE1/AE3) used for the primary antibody was dissolved in PBS containing 1% bovine serum albumin at a concentration described below.

Next, the tissue specimen t fixed to the slide is mounted in the sample chamber 1 of the immunohistochemical staining apparatus between the sample-side electrode 11 and opposed electrode 12, cytokeratin antibody (AE1/AE3) is dripped onto the section and stirred noncontactlessly by application of an alternating electric field that is adjusted so the opposed electrode 11 is minus with respect to the sample-side electrode 12. The antibody concentration at this time is 1.0 to 0.25 μg/mL, and the amount that is dripped onto the section is 150 to 200 μl. The alternating electric field is applied for 60 to 180 minutes. Electric field conditions are a voltage of 3.4 kV (3.0 to 3.8), an offset of 2.4 kV (2.0 to 2.8), a frequency of 18 Hz (±2), a distance of 6 mm between the field electrodes, a field strength of 0.533 kV/mm, 15-cycle bursts (15 to 30), and a square waveform.

Next, the cytokeratin antibody and the section antigen-antibody reacted by noncontact stirring is washed with PBS for 10 seconds, and the cytokeratin antibody (AE1/AE3) was removed. Then, 200 μl of the EnVision secondary antibody is dripped and noncontact stirring is carried out by applying for 2 minutes an alternating electric field adjusted so the opposed electrode 11 is minus with respect to the sample-side electrode 12. The antibody concentration at this time is as indicated in the EnVision (Doko Company) kit, and the electric field conditions are the same as when the primary antibody was applied.

The EnVision product and the section antigen-antibody reacted by noncontact stirring is then washed with PBS for 10 seconds, and the DAB chromogenic solution used to effect the EnVision color-development. Then, the microscope is used to observe the state of color development of the section in situ on the slide.

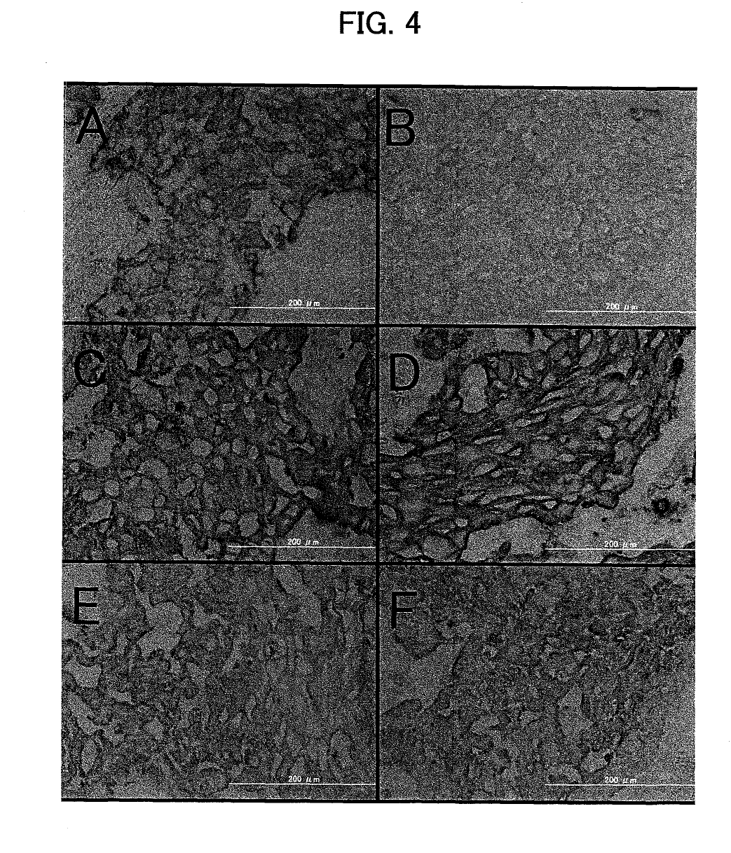

FIG. 4 shows the observation results.

2) Rapid Method

In the rapid method, the section fixed to the slide is mounted in the sample chamber 1 of the immunohistochemical staining apparatus between the sample-side electrode 11 and opposed electrode 12. From then until when the cytokeratin antibody (AE1/AE3) is dripped onto the section, the process steps are the same as those used in the antibody economy method as described in the above 1).

Noncontact stirring is then carried out by applying an alternating electric field that is adjusted so that the opposed electrode 11 is minus with respect to the sample-side electrode 12. From 150 to 200 μl of the antibody solution having an antibody concentration of 5.0 μg/mL is dripped onto the section. The alternating electric field is applied for 2 minutes. The electric field conditions are the same as those used in the antibody economy method as described in the above 1).

Subsequent process steps are the same as those used in the antibody economy method as described in the above 1). Observation results are shown in FIG. 4.

As a control, a conventional method was used to carry out an antigen-antibody reaction using a primary antibody having a primary antibody concentration of 5.0 μg/mL by being left to stand for 60 minutes, and an antigen-antibody reaction using the EnVision product by being left to stand for 30 minutes. The observation results are also shown in FIG. 4.

Details of the photographs marked A to F in FIG. 4 are listed in Table 1.

TABLE 1

| | | Primary antibody | | Primary antibody | EnVision product | |
|---|---|---|---|---|---|---|
| Photo | Staining Method | concentration | | (condition/time) | (condition/time) | Decision |
| A | Conventional | 5 | μg/mL | Standing/60 minutes | Standing/30 minutes | Positive |
| B | Rapid method control | 5 | μg/mL | Standing/2 minutes | Standing/2 minutes | Negative |
| C | Rapid method | 5 | μg/mL | Electric field/2 minutes | Electric field/2 minutes | Positive |
| D | Antibody economy method | 1 | μg/mL | Electric field/60 minutes | Electric field/2 minutes | Positive |
| E | Antibody economy method | 0.5 | μg/mL | Electric field/60 minutes | Electric field/2 minutes | Positive |
| F | Antibody economy method | 0.25 | μg/mL | Electric field/180 minutes | Electric field/2 minutes | Positive |

With respect to the observation results, as shown in Table 1 corresponding to FIG. 4, Photo A shows that cancer cells were stained by a conventional method comprising an antigen-antibody reaction using a primary antibody having a primary antibody concentration of 5.0 μg/mL carried out by being left to stand for 60 minutes, and an antigen-antibody reaction using the EnVision product carried out by being left to stand for 30 minutes. Photo B shows that cancer cells were not stained by a method, as a negative control of the above rapid method as described in the above 1), comprising an antigen-antibody reaction using a primary antibody having a primary antibody concentration of 5.0 μg/mL carried out by being left to stand for 2 minutes, and an antigen-antibody reaction using the EnVision product carried out by being left to stand for 2 minutes. Photo C shows that cancer cells were stained in accordance with the rapid method as described in the above 2) comprising an antigen-antibody reaction using a primary antibody having a primary antibody concentration of 5.0 μg/mL carried out in an electric field for 2 minutes, and an antigen-antibody reaction using the EnVision product carried out in an electric field for 2 minutes. Photos D to F show that cancer cells were stained to a level allowing a positive determination in accordance with the antibody economy method as described in the above 1) comprising an antigen-antibody reaction using a primary antibody carried out in an electric field for 60 to 180 minutes, and an antigen-antibody reaction using the EnVision product carried out in an electric field for 2 minutes, although the change in primary antibody concentration from 1 μg/mL to 0.25 μg/mL in the course of these gradually weakened the DAB color development.

The sequence of operations for immunohistochemical staining by the rapid method as described in the above 2) can be completed in 30 minutes, especially in 21 minutes, so it can readily clear the time constraints imposed by rapid intraoperative pathological diagnosis.

Therefore, with the immunohistochemical staining apparatus and immunohistochemical staining method according to this invention, an alternating electric field is applied to a tissue specimen mounted between a sample-side electrode and an opposed electrode. The alternating electric field is adjusted so that the opposed electrode is minus with respect to the sample-side electrode, enabling the antigen-antibody reaction between the tissue specimen and the primary and secondary antibodies to proceed under noncontact stirring generated by the Coulomb force of the alternating electric field which gives rise to molecular collisions, thereby making it possible to expedite the antigen-antibody reaction, as in the case of the rapid method as described in the above 2), making it applicable to rapid intraoperative pathological diagnoses. The antigen-antibody reaction between the tissue specimen and the primary and secondary antibodies that proceeds under noncontact stirring generated by the Coulomb force of the alternating electric field makes it possible to implement good immunohistochemical staining, even when, as in the case of the antibody economy method as described in the above 1), the amount of primary antibody used is one-tenth the amount used by a conventional method. This provides added-value with respect to environmental considerations because the amount of the used solution to be treated after carrying out the methods will reduce.

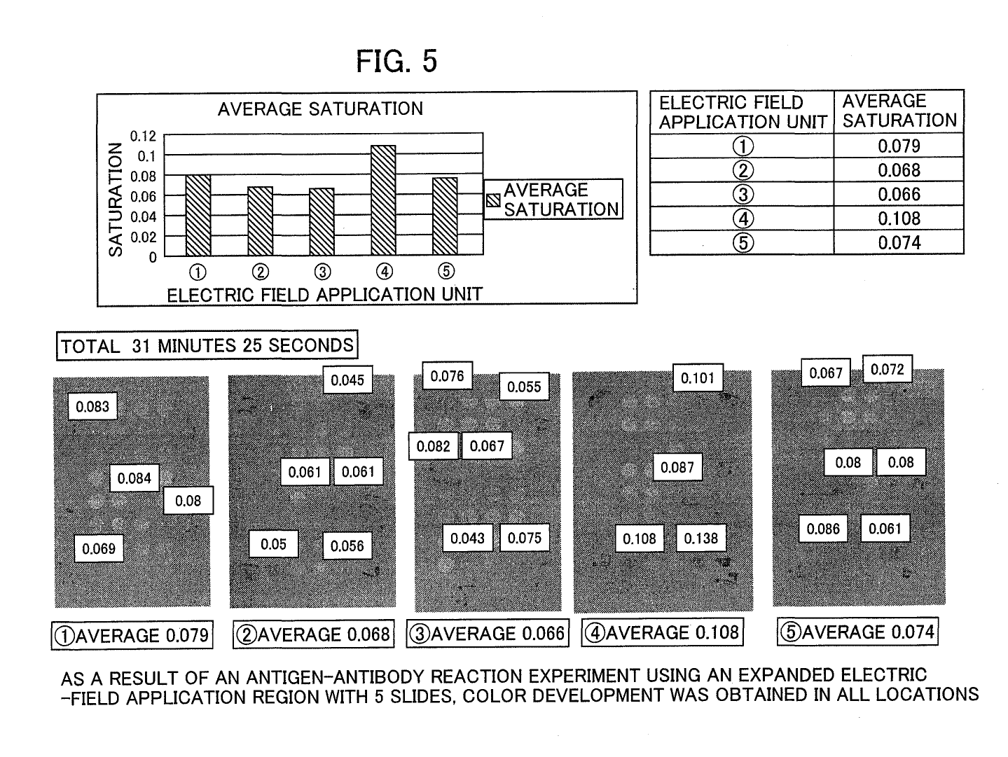

The immunohistochemical staining apparatus of the invention can be configured to accommodate five slides in the sample, for example, in the sample chamber shown in FIG. 5, making it possible to process simultaneously a plurality of sample specimens in a single operation. Specifically, the configuration is one in which a plurality of slides is arrayed between a pair of electrodes to enable a plurality of sample specimens to be processed simultaneously by the application of an alternating electric field. It is also possible to divide the sample chamber into a plurality of compartments, provide each with electrodes, and mount slides into the compartments one by one and apply an alternating electric field. As shown in FIG. 5, color development was confirmed in all of the samples on the five slides.

This makes it possible to provide an apparatus that can cope with the demands of a clinical setting by being able to handle the processing of multiple slides, as required for cytodiagnoses used in rapid intraoperative pathological diagnoses.

In the foregoing the present invention has been described with reference to various embodiments. However, the invention is not limited to the above embodiments and may be modified or otherwise changed in any way that does not depart from the scope of the claims. As the component elements of the invention, such as for example component parts of the apparatus and equipment required for implementation, there may be used elements that are well-known or widely known, or improved versions thereof.

When the invention is used for rapid intraoperative pathological diagnosis, as in the above embodiments, it goes without saying that it is not limited to the detection of lymph node microcarcinoma but can also be utilized in rapid intraoperative pathological diagnoses of liver cancer, breast cancer, colon cancer, and other cancers of the digestive organs such as cancer of the esophagus. When it is used for the antibody economy method, it can be applied virtually without exception to immunohistochemical staining carried out in general fields of medicine, dentistry and pharmacology, and biological research and development.

### INDUSTRIAL APPLICABILITY

With the immunohistochemical staining technology according to this invention, the antigen-antibody reaction can be energized by noncontact stirring, making it applicable to rapid intraoperative pathological diagnosis, which has strict time constraints. Also, since the antigen-antibody reaction can be energized by the noncontact stirring, the amount of antibody used in the immunohistochemical staining can be markedly reduced, making it possible to reduce the cost of the immunohistochemical staining method which is said to depend on the price of the primary antibody.

If the first prescribed time period for which the alternating electric field is applied is 1 to 5 minutes, and/or the second prescribed time period for which the alternating electric field is applied is 30 seconds to 5 minutes, the invention can be applied to rapid intraoperative pathological diagnoses, which has strict time constraints. The invention therefore has very high industrial applicability.

What is claimed is:

1. An immunohistochemical staining method, comprising:
forming a primary sample by applying a primary antibody to a tissue specimen disposed between a sample-side electrode and an opposed electrode, and noncontactlessly stirring the primary sample by applying for a first prescribed time period an alternating electric field adjusted so that the opposed electrode is minus with respect to the sample-side electrode;
then forming a secondary sample by applying a labeled secondary antibody to the primary sample, and noncontactlessly stirring the secondary sample by applying for a second prescribed time period an alternating electric field adjusted so that the opposed electrode is minus with respect to the sample-side electrode.

2. An immunohistochemical staining method according to claim 1, wherein the alternating electric field is a square wave superposed with a signal of a frequency of from 10 Hz to 300 Hz.

3. An immunohistochemical staining method according to claim 1 or 2, wherein a field strength of the alternating electric field is from 0.35 kV/mm to 2.50 kV/mm.

4. An immunohistochemical staining method according to any one of claims 1 to 3, wherein the label is any one selected from among an enzyme label, a fluorescence label, a radioisotope label, and a gold colloid particle label.

5. An immunohistochemical staining method according to any one of claims 1 to 4, wherein the first prescribed time period for which the alternating electric field is applied is 60 minutes to 180 minutes, and/or the second prescribed time period for which the alternating electric field is applied is 30 seconds to 5 minutes.

6. An immunohistochemical staining method according to any one of claims 1 to 5, wherein the primary antibody that is applied has a concentration of 0.25 μg/mL to 1.0 μg/mL.

7. An immunohistochemical staining method according to any one of claims 1 to 4, wherein the first prescribed time period for which the alternating electric field is applied is 1 minute to 5 minutes, and/or the second prescribed time period for which the alternating electric field is applied is 30 seconds to 5 minutes.

8. An immunohistochemical staining method according to any one of claims 1 to 4 and 7, wherein the primary antibody that is applied has a concentration of 4.0 μg/mL to 6.0 μg/mL.

9. An immunohistochemical staining apparatus, comprising:
a sample chamber in which a primary sample comprised of a tissue specimen to which a primary antibody is applied is disposed between a sample-side electrode and an opposed electrode;
the sample chamber including first stirring means for noncontactlessly stirring the primary sample by applying for a first prescribed time period an alternating electric field adjusted so that the opposed electrode is minus with respect to the sample-side electrode, and second stirring means for noncontactlessly stirring a secondary sample comprised of the noncontactlessly-stirred primary sample to which a labeled secondary antibody is applied by applying for a second prescribed time period an alternating electric field adjusted so that the opposed electrode is minus with respect to the sample-side electrode.

10. An immunohistochemical staining apparatus according to claim 9, wherein the alternating electric field is a square wave superposed with a signal of a frequency of from 10 Hz to 300 Hz.

11. An immunohistochemical staining apparatus according to claim 9 or 10, wherein a field strength of the alternating electric field is from 0.35 kV/mm to 2.50 kV/mm.

12. An immunohistochemical staining apparatus according to any one of claims 9 to 11, wherein the label is any one selected from among an enzyme label, a fluorescence label, a radioisotope label, and a gold colloid particle label.

13. An immunohistochemical staining apparatus according to any one of claims 9 to 12, wherein the first prescribed time period for which the alternating electric field is applied is 60 minutes to 180 minutes, and/or the second prescribed time period for which the alternating electric field is applied is 30 seconds to 5 minutes.

14. An immunohistochemical staining apparatus according to any one of claims 9 to 13, wherein the primary antibody that is applied has a concentration of 0.25 μg/mL to 1.0 μg/mL.

15. An immunohistochemical staining apparatus according to any one of claims 9 to 12, wherein the first prescribed time period for which the alternating electric field is applied is 1 minute to 5 minutes, and/or the second prescribed time period for which the alternating electric field is applied is 30 seconds to 5 minutes.

16. An immunohistochemical staining apparatus according to any one of claims 9 to 12 and 15, wherein the primary antibody has a concentration of 4.0 μg/mL to 6.0 μg/mL.

17. An immunohistochemical staining apparatus according to any one of claims 9 to 16, wherein the first stirring means and the second stirring means are identical.

18. An immunohistochemical staining apparatus according to any one of claims 9 to 17, wherein a part of the opposed electrode that is directly above the tissue specimen mounted in the sample chamber is provided with a projecting portion.
